(19) United States
(12) Patent Application Publication
(10) Pub. No.: US 2024/0092751 A1
(43) Pub. Date: Mar. 21, 2024

(54) NEW BUTYRATE COMPOUNDS

(71) Applicant: DSM IP Assets B.V., Heerlen (NL)

(72) Inventors: Werner BONRATH, Kaiseraugst (CH); Jonathan Alan MEDLOCK, Kaiseraugst (CH); Francesco PACE, Kaiseraugst (CH)

(21) Appl. No.: 18/261,147

(22) PCT Filed: Jan. 28, 2022
(86) PCT No.: PCT/EP2022/052003

§ 371 Date: Jul. 12, 2023

(30) Foreign Application Priority Data
Feb. 1, 2021 (EP) 21154606.4

(51) Int. Cl. C07D 311/72

(52) U.S. Cl. CPC C07D 311/72

(57) ABSTRACT

The present invention relates to butyrate compounds as well as to their synthesis and their use. Such compounds are very useful compounds, either as such or as intermediates in organic synthesis.

The present invention relates to new butyrate compounds as well as to their synthesis and their use. Such compounds are very useful compounds, either as such or as intermediates in organic synthesis.

Butyrates are seen as very useful and healthy compounds.

It is known that butyrates fuels colonocytes, and in return these cells help provide an oxygen-free environment in which beneficial gut microbes thrive. This keeps inflammation in check, gut cells healthy, and gut bacteria in a good state.

Higher butyrate levels have been shown to increase levels of glutathione, an antioxidant produced in the body's cells which neutralises free radicals in the gut. This is good because free radicals are linked to inflammation and many diseases.

Butyrates stop some of the pro-inflammatory substances in the human body from working. The anti-inflammatory effect of butyrate reduces oxidative stress and controls the damage caused by free radicals.

Furthermore, research shows that butyrates enhance the secretion of gut hormones like glucagon-like peptide-1 (GLP-1) and peptide YY (PYY). GLP-1 increases insulin production and reduces glucagon production in the pancreas. PYY increases the uptake of glucose in both muscles and fatty tissue.

Increased production of short-chain fatty acids, including butyrate in the colon, increases the release of these gut hormones, indicating potential benefits for managing blood sugar levels and preventing weight gain.

Furthermore, butyrates can be used as intermediates in organic synthesis to produce i.e. useful carotenoid compounds.

The main problem with the butyrates is the strong (fishy) odour. Such an odour is such that most persons are not able to swallow such a compound even in very low concentration.

Therefore, the goal of the present invention was to provide butyrate compounds having all advantages but not having the strong unpleasant odour.

Surprisingly, it was found that specific new butyrate compounds fulfil the requirements as listed above and not having the strong unpleasant odour.

Therefore, the present invention relates the compound of formula (I)

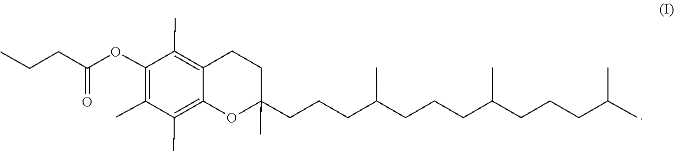 [SMILES: CCCC(=O)Oc1c(C)c(C)c2c(c1C)CCC(C)(CCCC(C)CCCC(C)CCCC(C)C)O2]

The new compound of formula (I) can be obtained as outlined below.

The starting material to obtain the compound of formula (I) is α-tocopherol, which is the compound of formula (II)

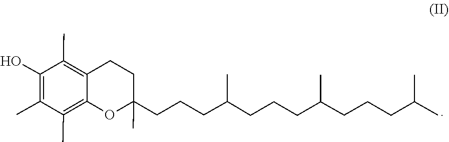 [SMILES: Cc1c(C)c2c(c(C)c1O)CCC(C)(CCCC(C)CCCC(C)CCCC(C)C)O2]

α-Tocopherol, which is a form of Vitamin E, is available easily. It is commercially available from a wide range of sources and suppliers. It can be chemically synthesized or extracted from natural sources.

The compound of formula (I) is produced by a two-step process.

In a first step (step (i)), the compound of formula (II) is esterified by using the acid of formula (III), or a derivative thereof.

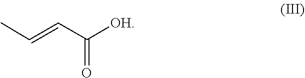

The advantage of this reaction is that the acid of formula (III) has no strong unpleasant odor, unlike the related compound butyric acid.

The reaction product of step (i) is the compound of formula (IV)

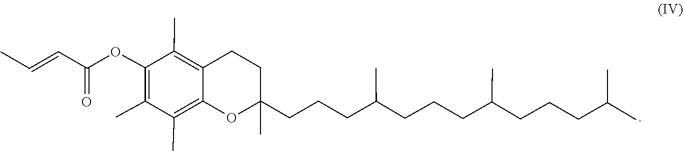 [SMILES: C/C=C/C(=O)Oc1c(C)c(C)c2c(c1C)CCC(C)(CCCC(C)CCCC(C)CCCC(C)C)O2]

The compound of formula (IV) is a new compound.

Therefore the present invention also relates to a compound of formula (IV)

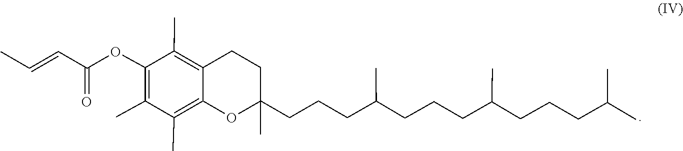 [SMILES: C/C=C/C(=O)Oc1c(C)c(C)c2c(c1C)CCC(C)(CCCC(C)CCCC(C)CCCC(C)C)O2]

In a second step (step (ii)) the compound of formula (IV) is hydrogenated to the compound of formula (I).

Therefore, the present invention relates to a process (P) to produce the compound of formula (I)

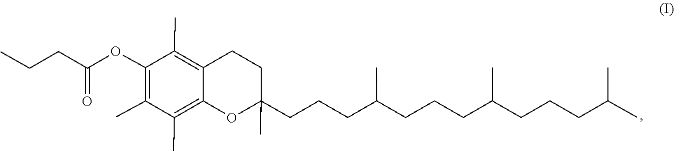 [SMILES: CCCC(=O)Oc1c(C)c(C)c2c(c1C)CCC(C)(CCCC(C)CCCC(C)CCCC(C)C)O2]

wherein in a first step (step (i)), the compound of formula (II)

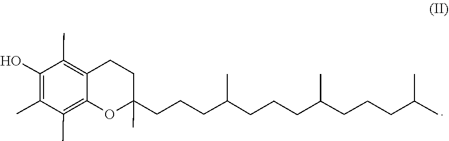 [SMILES: Cc1c(C)c2c(c(C)c1O)CCC(C)(CCCC(C)CCCC(C)CCCC(C)C)O2]

is esterified by the compound of formula (III) (or a derivative thereof)

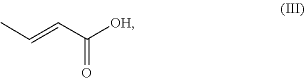

and then in a second step (step (ii)) the reaction product of step (i) is hydrogenated to the compound of formula (I).

Some of the advantages of the process are that a good yield is obtained and that no unpleasant smelling starting material are used and no unpleasant smelling products are obtained.

In the following the process steps are discussed in more details.

### Step (i)

As stated above the compound of formula (II)

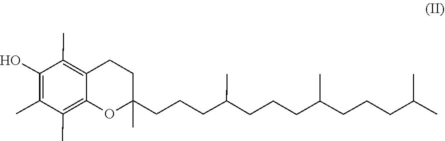 [SMILES: Cc1c(C)c2c(c(C)c1O)CCC(C)(CCCC(C)CCCC(C)CCCC(C)C)O2]

is esterified by using the acid of formula (III)

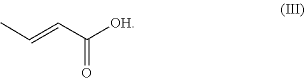

The reaction of step (i) is usually carried out in an inert solvent (or in a mixture of inert solvents). Such inert solvents can be aliphatic or aromatic.

Suitable solvents include hydrocarbons, chlorinated hydrocarbons, ethers, amides and amines. Preferred are hydrocarbons, chlorinated hydrocarbons, ethers and amides.

It is also possible to carry out the process of step (i) without any inert solvent.

Therefore, the present invention relates to a process (P1), which relates to the process (P), wherein the process of step (i) is carried out in at least one inert solvent.

Therefore, the present invention relates to a process (P1′), which relates to the process (P1), wherein the at least solvent is aliphatic or aromatic.

Therefore, the present invention relates to a process (P1″), which relates to the process (P), wherein the at least one inert solvent is chosen from the group consisting of hydrocarbons, chlorinated hydrocarbons, ethers, amides and amines (preferred are hydrocarbons, chlorinated hydrocarbons, ethers and amides).

Therefore, the present invention relates to a process (P1′″), which relates to the process (P), wherein the process of step (i) is carried without any solvent.

The process of step (i) is usually and preferably carried out at elevated temperatures. The reaction temperature of step (i) is usually from 30° C. to 150° C. Preferably 40° C. to 140° C., more preferably 50° C. to 130° C.

Therefore, the present invention relates to a process (P2), which relates to the process (P), (P1), (P1′), (P1″) or (P1′″) wherein the process of step (i) is carried out at elevated temperatures.

Therefore, the present invention relates to a process (P2′), which relates to the process (P2), wherein the process of step (i) is carried out at a temperature of 30° C. to 150° C.

Therefore, the present invention relates to a process (P2″), which relates to the process (P2), wherein the process of step (i) is carried out at a temperature of 40° C. to 140° C.

Therefore, the present invention relates to a process (P2′″), which relates to the process (P2), wherein the process of step (i) is carried out at a temperature of 50° C. to 130° C.

The process of step (i) is preferably carried out in the presence of at least one additional acid (in addition to the compound of formula (III)).

The additional acid can be organic as well as inorganic as well as solid as well a heteropolyacid.

Furthermore, the additional acid can be a solid acid, such as acid ion exchange resins (i.e. example AMBERLYST types), as well as a heteropolyacids (such as H₃PW₁₂O₄₀).

Suitable inorganic acids are i.g. H₂SO₄, HCl, preferably H₂SO₄.

Suitable organic acids are i.g. methanesulfonic acid, p-toluenesulfonic acid and trifluoroacetic acid.

Therefore, the present invention relates to a process (P3), which relates to the process (P), (P1), (P1′), (P1″), (P1′″), (P2), (P2′), (P2″) or (P2′″), wherein the process of step (i) is carried out in the presence of at least one additional acid.

Therefore, the present invention relates to a process (P3′), which relates to the process (P3), wherein the at least one additional acid is organic.

Therefore, the present invention relates to a process (P3″), which relates to the process (P3′), wherein the additional acid is chosen from the group consisting of methanesulfonic acid, p-toluenesulfonic acid and trifluoroacetic acid.

Therefore, the present invention relates to a process (P3′″), which relates to the process (P3), wherein the at least one additional acid is inorganic.

Therefore, the present invention relates to a process (P3″″), which relates to the process (P3′″), wherein the additional acid is chosen from the group consisting of HCl and H₂SO₄.

Therefore, the present invention relates to a process (P3′″″), which relates to the process (P3), wherein the additional acid is chosen from the group consisting of solid acids (such as ion exchange resins).

Therefore, the present invention relates to a process (P3″″″), which relates to the process (P3), wherein the additional acid is chosen from the group consisting of heteropolyacids (such as H₃PW₁₂O₄₀).

The at least one additional acid is usually added in an amount of 1-20 mol-% (in regard to compound of formula (II)).

Therefore, the present invention relates to a process (P3′″″″), which relates to the process (P3), (P3′), (P3″), (P3′″), (P3″″),(P3′″″) or (P3″″″), wherein the at least one additional acid is added in an amount of 1-20 mol-% (in regard to compound of formula (II)).

The compound of formula (III) is added preferably in an excess in view of compound of formula (II). Usually the molar ratio of compound of formula (II) to compound of formula (III) is 1:1 to 1:10 (preferably 1:2 to 1:6).

Therefore, the present invention relates to a process (P4), which relates to the process (P), (P1), (P1′), (P1″), (P1′″), (P2), (P2′), (P2″), (P2′″), (P3), (P3′), (P3″), (P3′″), (P3″″), (P3′″″), (P3″″″) or (P3′″″″), wherein the molar ratio of compound of formula (II) to compound of formula (III) is 1:1 to 1:10.

Therefore, the present invention relates to a process (P4′), which relates to the process (P), (P1), (P1′), (P1″), (P1′″), (P2), (P2′), (P2″), (P2′″), (P3), (P3′), (P3″), (P3′″), (P3″″), (P3′″″), (P3″″″) or (P3′″″″), wherein the molar ratio of compound of formula (II) to compound of formula (III) is 1:2 to 1:6.

Usually the obtained reaction product of step (i), which is the compound of formula (IV), is isolated and optionally purified and then used in the second step (step (ii)).

### Step (ii)

As disclosed above in step (ii) of the process according to the present invention, the obtained reaction product of step (i) is hydrogenated to obtain the desired compound of formula (I).

Usually and preferably H₂ gas is used to hydrogenate the compound of formula (IV).

Therefore, the present invention relates to a process (P5), which relates to the process (P), (P1), (P1′), (P1″), (P1′″), (P2), (P2′), (P2″), (P2′″), (P3), (P3′), (P3″), (P3′″), (P3″″), (P3′″″), (P3″″″) or (P3′″″″), (P4) or (P4′), wherein the process of step (ii) H₂ gas is used for the hydrogenation.

The pressure applied in the process of step (ii) is usually (and preferably) higher than 1.5 bar. The pressures given here are absolute. Usually (and preferably) the applied pressure is in the range of 1.5 to 11 bar (more preferably 2 to 10 bar).

Therefore, the present invention relates to a process (P6), which relates to the process (P), (P1), (P1′), (P1″), (P1′″), (P2), (P2′), (P2″), (P2′″), (P3), (P3′), (P3″), (P3′″), (P3″″), (P3′″″), (P3″″″) or (P3′″″″), (P4), (P4′) or (P5), wherein the pressure applied in the process of step (ii) is higher than 1.5 bar.

Therefore, the present invention relates to a process (P6′), which relates to the process (P), (P1), (P1′), (P1″), (P1′″), (P2), (P2′), (P2″), (P2′″), (P3), (P3′), (P3″), (P3′″), (P3″″), (P3′″″), (P3″″″) or (P3′″″″), (P4), (P4′) or (P5), wherein the pressure applied in the process of step (ii) is in the range of 1.5 to 11 bar.

Therefore, the present invention relates to a process (P6″), which relates to the process (P), (P1), (P1′), (P1″), (P1′″), (P2), (P2′), (P2″), (P2′″), (P3), (P3′), (P3″), (P3′″), (P3″″), (P3′″″), (P3″″″), (P3′″″″), (P4), (P4′) or (P5), wherein the pressure applied in the process of step (ii) is in the range of 2 to 10 bar.

The hydrogenation is usually carried out in the presence of a catalyst.

The catalyst can be any commonly used catalyst for hydrogenation reactions. Usually it is a metal catalyst (preferably a transition metal catalyst).

Preferably it is a heterogeneous catalyst comprising at least one of metal chosen from the group consisting of nickel, palladium or platinum.

Therefore, the present invention relates to a process (P7), which relates to the process (P), (P1), (P1′), (P1″), (P1′″), (P2), (P2′), (P2″), (P2′″), (P3), (P3′), (P3″), (P3′″), (P3″″), (P3′″″), (P3″″″) or (P3′″″″), (P4), (P4′), (P5), (P6), (P6′) or (P6″), wherein the hydrogenation of step (ii) is carried out in the presence of a catalyst.

Therefore, the present invention relates to a process (P7′), which relates to the process (P7), wherein the catalyst is a metal catalyst.

Therefore, the present invention relates to a process (P7″), which relates to the process (P7), wherein the catalyst is a transition metal catalyst.

Therefore, the present invention relates to a process (P7′″), which relates to the process (P7), wherein the catalyst is a heterogeneous catalyst comprising at least one of metal chosen from the group consisting of nickel, palladium or platinum.

The process of step (ii) is usually (and preferably) carried out at elevated temperatures.

Usually (and preferably) the process of step (ii) is carried out at a temperature of 30° C. to 100° C. (preferably at a temperature of 30° C. to 80° C., more preferably at a temperature of 30° C. to 60° C.).

Therefore, the present invention relates to a process (P8), which relates to the process (P), (P1), (P1′), (P1″), (P1′″), (P2), (P2′), (P2″), (P2′″), (P3), (P3′), (P3″), (P3′″), (P3″″), (P3′″″), (P3″″″) or (P3′″″″), (P4), (P4′), (P5), (P6), (P6′), (P6″), (P7), (P7′), (P7″) or (P7′″), wherein the process of step (ii) is carried out at an elevated temperature.

Therefore, the present invention relates to a process (P8′), which relates to the process (P), (P1), (P1′), (P1″), (P1′″), (P2), (P2′), (P2″), (P2′″), (P3), (P3′), (P3″), (P3′″), (P3″″), (P3′″″), (P3″″″) or (P3′″″″), (P4), (P4′), (P5), (P6), (P6′), (P6″), (P7), (P7′), (P7″) or (P7′″), wherein the process of step (ii) is carried out at a temperature of 30° C. to 100° C.

Therefore, the present invention relates to a process (P8″), which relates to the process (P), (P1), (P1′), (P1″), (P1′″), (P2), (P2′), (P2″), (P2′″), (P3), (P3′), (P3″), (P3′″), (P3″″), (P3′″″), (P3″″″) or (P3′″″″), (P4), (P4′), (P5), (P6), (P6′), (P6″), (P7), (P7′), (P7″) or (P7′″), wherein the process of step (ii) is carried out at a temperature of 30° C. to 80° C.

Therefore, the present invention relates to a process (P8′″), which relates to the process (P), (P1), (P1′), (P1″), (P1′″), (P2), (P2′), (P2″), (P2′″), (P3), (P3′), (P3″), (P3′″), (P3″″), (P3′″″), (P3″″″) or (P3′″″″), (P4), (P4′), (P5), (P6), (P6′), (P6″), (P7), (P7′), (P7″) or (P7′″), wherein the process of step (ii) is carried out at a temperature of 30° C. to 60° C.

The reaction of step (ii) is usually carried out in an inert solvent (or in a mixture of inert solvents). Such inert solvents can be aliphatic or aromatic.

Suitable solvents include hydrocarbons, chlorinated hydrocarbons, ethers, esters and alcohols, amides and amines.

It is also possible to carry out the process of step (i) without any inert solvent.

Therefore, the present invention relates to a process (P9), which relates to the process (P), (P1), (P1′), (P1″), (P1′″), (P2), (P2′), (P2″), (P2′″), (P3), (P3′), (P3″), (P3′″), (P3″″), (P3′″″), (P3″″″) or (P3′″″″), (P4), (P4′), (P5), (P6), (P6′), (P6″), (P7), (P7′), (P7″), (P7′″), (P8), (P8′), (P8″) or (P8′″), wherein the process of step (ii) is carried out in at least one inert solvent.

Therefore, the present invention relates to a process (P9′), which relates to the process (P9), wherein the at least solvent is aliphatic or aromatic.

Therefore, the present invention relates to a process (P9″), which relates to the process (P9), wherein the at least one inert solvent is chosen from the group consisting hydrocarbons, chlorinated hydrocarbons, ethers, esters and alcohols, amides and amines.

Therefore, the present invention relates to a process (P9′″), which relates to the process (P9), wherein the process of step (ii) is carried without any solvent.

The reaction product of step (ii) is the final product, which is the compound of formula (I). The compound of formula (I) can be used as obtained from step (ii) or can be purified by standard techniques to the required quality.

The compound of formula (I) can be used as such (or also in a formulation) or it can also be used as an intermediate in organic synthesis.

The following Example illustrates the invention further without limiting it. All percentages and parts, which are given, are related to the weight and the temperatures are given in ° C., and the pressures are absolute pressures when not otherwise stated.

### EXAMPLES

### Example 1

### Step (i)

In a 50-ml four-necked flask equipped with a KPG-stirrer, thermometer, water separator and a reflux condenser with an argon inlet, 4.8 g (11.15 mmol) (all-rac)-α-tocopherol and 4.12 g (47.8 mmol) crotonic acid were dissolved in 40 ml toluene in the presence of 94 μl (15 mol%, 1.696 mmol) H₂SO₄ (96.7%). The mixture was stirred at 400 rpm and heated at 383 K (internal temperature) for 18 h. The mixture was dissolved in 40 ml toluene and washed 3 times with 20 ml H₂O and dried with sodium sulfate and evaporated under reduced pressure (10 mbar, 313 K). The crude product was further purified 2 times by bulb-to-bulb distillation. The first one at 393 K (0.03 mbar) for separate the excess of crotonic acid and the second time at 533 K (0.03 mbar) for the product. The pure product was isolated as colorless-light yellowish oil in 93.8% purity (LC-Area).

Yield 5.52 g (all-rac)-α-tocopheryl crotonate, 93% based on (all-rac)-α-tocopherol).

### Step (ii)

In a 50-ml flask equipped with a magnetic-stirrer, 4.2 g (8.19 mmol) (all-rac)-α-tocopheryl-crotonate, 320 mg catalyst 5% Pd/Al₂O₃ and 22 g n-heptane were mixed together. The reaction mixture was purged 3 times with nitrogen (pressurise to 6 bar and release). The mixture was heated to 313 K and then pressurised to 6 bar with hydrogen gas. The mixture was stirred at 1000 rpm at 313 K jacket temperature for 2 h. The mixture was cooled to room temperature and the pressure was released. The catalyst was removed by filtration and the filtrate was evaporated under reduced pressure (10 mbar, 313 K). The crude product was isolated as colorless-light yellowish oil in 98.7% purity (GC-Area).

Yield 4.13 g (all-rac)-α-tocopheryl-butyrate, 99% based on (all-rac)-α-tocopheryl-crotonate.

1. Process to produce the compound of formula (I) 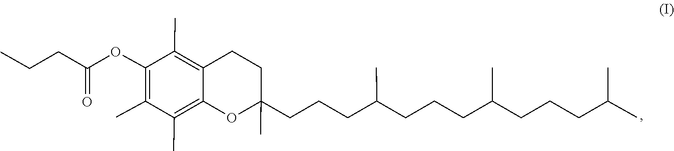 [SMILES: CCCC(=O)Oc1c(C)c(C)c2c(c1C)CCC(C)(CCCC(C)CCCC(C)CCCC(C)C)O2]
wherein in a first step (step (i)), the compound of formula (II) 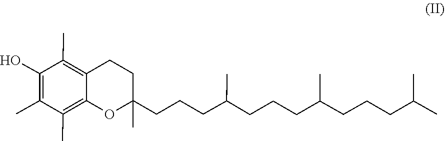 [SMILES: Cc1c(C)c2c(c(C)c1O)CCC(C)(CCCC(C)CCCC(C)CCCC(C)C)O2]
is esterified by the compound of formula (III) (or a derivative thereof) 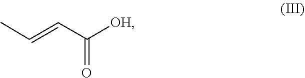
and then in a second step (step (ii)) the reaction product of step (i) is hydrogenated to the compound of formula (I).

2. Process according to claim 1, wherein the process of step (i) is carried out in at least one inert solvent.

3. Process according to claim 2, wherein the at least solvent is aliphatic or aromatic.

4. Process according to claim 1, wherein the process of step (i) is carried out without any inert solvent.

5. Process according to claim 1, wherein the process of step (i) is carried out at elevated temperatures.

6. Process according to claim 4, wherein the process is carried out at a temperature of 30° C. to 150° C.

7. Process according to claim 1, wherein the process of step (i) is carried out in the presence of at least one additional acid.

8. Process according to claim 7, wherein the at least one additional acid is organic or inorganic.

9. Process according to claim 7, wherein the at least one additional acid is added in an amount of 1-20 mol-% (in regard to compound of formula (II)).

10. Process according to claim 1, wherein the molar ratio of compound of formula (II) to compound of formula (III) is 1:1 to 1:10.

11. Process according to claim 1, wherein the process of step (ii) H₂ gas is used for the hydrogenation.

12. Process according to claim 11, wherein the pressure applied in the process of step (ii) is higher than 1.5 bar.

13. Process according to claim 1, wherein the hydrogenation of step (ii) is carried out in the presence of a catalyst.

14. Compound of formula (I)

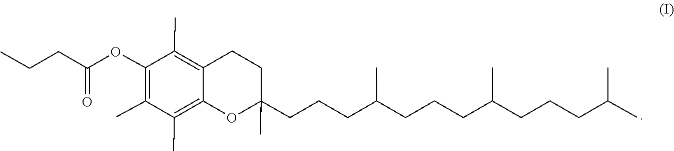 [SMILES: CCCC(=O)Oc1c(C)c(C)c2c(c1C)CCC(C)(CCCC(C)CCCC(C)CCCC(C)C)O2]

15. Compound of formula (IV) 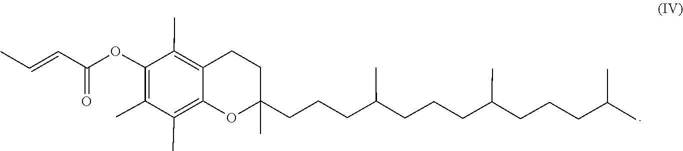 [SMILES: C/C=C/C(=O)Oc1c(C)c(C)c2c(c1C)CCC(C)(CCCC(C)CCCC(C)CCCC(C)C)O2]
